(19) United States
(12) Patent Application Publication
(10) Pub. No.: US 2019/0083636 A1
(43) Pub. Date: Mar. 21, 2019

(54) METHODS OF TREATING DISEASES RELATED TO NET FORMATION WITH PARENTERAL ADMINISTRATION OF POLYSIALYLATED DNASE I

(71) Applicant: Lipoxen Technologies Limited, London (GB)

(72) Inventor: Dmitry Genkin, St. Petersburg (RU)

(73) Assignee: Lipoxen Technologies Limited, London (GB)

(21) Appl. No.: 15/962,636

(22) Filed: Apr. 25, 2018

Related U.S. Application Data
(60) Provisional application No. 62/489,915, filed on Apr. 25, 2017.

(51) Int. Cl. A61K 47/61; A61K 38/46; A61K 9/00

(52) U.S. Cl. CPC A61K 47/61; A61K 38/465; C12Y 301/21001; A61K 9/007; A61K 9/0019

(57) ABSTRACT

The present invention provides conjugates of deoxyribonuclease enzymes with water soluble polymers such as PSA having improved pharmacokinetic attributes. These modifications provide unexpectedly high levels of DNA hydrolytic activity in blood and other bodily tissues over the time due to markedly increased distribution phase and reduced clearance of DNase conjugates after delivery to blood circulation relative to the unconjugated compounds, while half-life and residence time of conjugates remains almost unchanged compared to the unconjugated DNase compounds. The compositions of the invention are used for parenteral treatment of diseases related to NET formation and the presence of extracellular DNA.

### RELATED APPLICATIONS

This application claims priority and is entitled to the filing date of U.S. provisional application Ser. No. 62/489,915 filed on Apr. 25, 2017. The contents of the aforementioned application(s) are incorporated herein by reference.

### BACKGROUND

Neutrophil extracellular traps (NETs) were discovered as extracellular strands of decondensed DNA, which were expelled from activated neutrophils. NETs have been implicated as key players into pathogenesis of an increasingly large number of human diseases including cancer, acute organ injury, kidney disease, GVH disease, stroke, thrombosis, autoimmunity, diabetes, atherosclerosis, sepsis, eclampsia, fertility, coagulopathies and neurodegeneration. Endogenous deoxyribonuclease I (DNase I) enzyme activity is heavily suppressed in diseases accompanied by intensive NETs formation. It was discovered that DNase I can effectively degrade established NETs, thereby abolishing their pathogenic effect.

Cancers predispose neutrophils to release extracellular DNA traps that contribute to cancer-associated thrombosis and tumor progression. For example, studies have shown that administration of DNase is effective in metastatic pancreatic cancer to reduce coagulopathies and organ failure caused in part by DNA traps. Similar studies have shown efficacy in colorectal cancer, lung cancer, hepatoma, and metastases of these cancers.

For example, rhDNase I suppresses the development of metastatic disease by 60-70% when administered three times a week at 0.04-2 mg/kg in metastatic pancreatic cancer disease models. Administering rhDNase I restored perfusion in the kidney and heart and also prevented vessel leakage in the blood vasculature in animals with pancreatic cancer. Increased postoperative NET formation in colorectal cancer patients was associated with a >4-fold reduction in disease-free survival. rhDNase I treatment lead to 68% reduction in metastasis and a significant decrease in proliferation and angiogenesis in primary tumor in colorectal cancer models. rhDNase I also suppresses the development of metastatic disease by 60-90% when administered daily at 0.02-2.3 mg/kg in lung carcinoma and hepatoma metastatic disease model.

Early initiation of thrombolytic therapy is critical for stroke severity and outcome; National Institutes of Health (NIH) recommendation of “door to drug” on admission is within 60 minutes. However, IV tPA (Tissue Plasminogen Activator) can't be initiated prior CT, lab and neurological examination due to bleeding concerns. As the result only 5% of patients received medical treatment with the tPA due to short available window (4h since onset). NETs are involved into set up and growth of clot matrix, activation of platelets and coagulation cascade and induction of endothelial dysfunction; NETs trigger secondary brain injury after reperfusion. Both early and late treatment with recombinant human DNase 1 significantly improved IBS outcome. rhDNase I suppress clot formation and speed up clot dissolution without affecting blood coagulation cascade.

Studies demonstrate that DNase I treatment can reduce GVHD mortality and morbidity in mice. In comparison to PBS control, DNase I-treated mice showed a lower mortality rate. Within 4 weeks post-transplantation, 80% of DNase I-treated recipients survived, compared with only 30% of PBS control mice. Whereas control recipients had severe GVHD in the skin, intestine, liver, and lung, DNase I-treated mice exhibited only mild changes in these organs, reflected in their significantly lower GVHD scores.

Intravascular NETs release provoke vascular injury and tubular necrosis in acute kidney injury AKI). rhDNase attenuated sepsis-induced organ damage and improved the survival rate in CLP sepsis model. rhDNase administration every in CLP sepsis model also rescued mice from death and kidney/lung failure. In ischemia/reperfusion-induced AKI renal perfusion was significantly improved in rhDNase-treated animals, concomitantly with significant amelioration of damage to renal functioning and tissue integrity.

Deoxyribonuclease enzyme is thus a useful therapeutic compound to treat pathologic conditions related to increased amount of circulating cell free DNA and neutrophil extracellular traps. However, pharmacokinetic properties of natural deoxyribonuclease enzymes limit their therapeutic efficacy due to inability to maintain meaningful DNA hydrolytic activity in blood. Industrial applicability of natural deoxyribonuclease enzymes also limited since the quantities of enzyme required to maintain meaningful DNA hydrolytic activity in blood makes such treatment non-compliant to the patient and economically unfeasible.

Xenetic Biosciences has developed an inhalable formulation of second generation DNase I enzyme based on Xenetic polysialic acid technology for treatment of cystic fibrosis (PulmoXEN™). Continuous DNase I hydrolytic activity in sputum above equivalent of 0.1 mkg/ml was required for NET elimination and biofilm disruption. Pulmozyme activity declined very rapidly and disappears within 2 hours following inhalation. Polysialic acid conjugated rhDNase I was developed to address the issue. PulmoXEN™ showed delay of systemic absorption, better sputum penetration, less sensitivity to actin inhibition, increased stability, and expectorant activity of sialic acid (see, e.g., U.S. Pat. No. 8,981,050).

Polysialic acids (PSAs) are naturally occurring unbranched polymers of sialic acid produced by certain bacterial strains and in mammals in certain cells. They can be produced in various degrees of polymerisation from n=about 80 or more sialic acid residues down to n=2 by limited acid hydrolysis or by digestion with neuraminidases, or by fractionation of the natural, bacterially derived forms of the polymer.

In recent years, the biological properties of polysialic acids, particularly those of the alpha-2,8 linked homopolymeric polysialic acid, have been exploited to modify the pharmacokinetic properties of protein and low molecular weight drug molecules. Polysialic acid derivatisation gives rise to dramatic improvements in circulating half-life for a number of therapeutic proteins including catalase and asparaginase, and also allows such proteins to be used in the face of pre-existing antibodies raised as an undesirable (and sometimes inevitable) consequence of prior exposure to the therapeutic protein. The alpha-2,8 linked polysialic acid offers an attractive alternative to PEG, being an immunologically invisible biodegradable polymer which is naturally part of the human body, and which degrades, via tissue neuraminidases, to sialic acid, a non-toxic saccharide.

The present invention thus provides polysaccharide conjugates or derivatives of DNase for the treatment of disease related to NET formation and extracellular DNA, such as stroke, metastatic cancer (pancreatic, lung, hepatoma, and colorectal), acute kidney injury, GVH disease, venous thromboembolism, atherosclerosis, liver failure, acute lung injury and pulmonary fibrosis, dry eye disease, Alzheimer's disease, and disseminated intravascular coagulation and other diseases as listed below. The derivatives are useful for improving the stability, pharmacokinetics and pharmacodynamics of DNase for enteral or parenteral administration, such as subcutaneous, intravenous, intraperitoneal, and intramuscular administration, etc.

### SUMMARY OF THE INVENTION

In one aspect, the present invention provides a therapeutic composition for enzymatic cleavage of circulating cell free DNA and neutrophil extracellular traps in blood, the composition comprising a deoxyribonuclease enzyme conjugated with a water soluble polymer, wherein the DNase I conjugate has a systemic clearance and apparent volume of distribution each at least 50% or lower as compared to DNase that is not conjugated with a water soluble polymer; and wherein the DNase is formulated for parenteral administration.

In one embodiment, the DNase is DNase I. In one embodiment, the DNase is conjugated to the water soluble polymer via a linking group. In one embodiment, the water soluble polymer is PEG, poly(2-ethyl 2-oxazoline), poly[oligo(ethylene glycol) methyl methacrylate], polyoxazoline, poly(N-(2-hydroxypropyl) methacrylamide, polyglycerol, poly(N-vinylpyrrolidone), polycarbonate, poly(carboxybetaine methacrylate), poly(sulfobetaine methacrylate) or poly(-methyacryloyloxyethyl phosphorylcholine). In one embodiment, the DNase is linked via an amine group at the N-terminus to a water soluble polymer comprising a polysaccharide. In one embodiment, the polysaccharide is selected from polysialic acid, heparin, dextran, dextrin, hydroxyethyl starch, hyaluronic acid or chondroitin sulphate. In one embodiment, the polysaccharide is polysialic acid. In one embodiment, the polysialic acid is attached to the N-terminus of DNase at the reducing terminal unit of the polysialic acid.

In one embodiment, the DNase I has at least 95% sequence identity to an amino acid sequence comprising Accession No. AAA63170.1, AAB00495.1 or CAC12813.1. In one embodiment, the DNase I has an amino acid sequence comprising Accession No. AAA63170.1, AAB00495.1 or CAC12813.1. In one embodiment, the DNase I has an amino acid sequence change in DNA binding domain leading to increased hydrolytic activity. In one embodiment, the DNase I has an amino acid sequence change in actin binding site leading to loss of actin inhibitory properties

In one aspect, the invention provides a method for treating a disease state associated with circulating cell free DNA and neutrophil extracellular traps in blood, lymph and synovial the method comprising parenteral administration to a subject in need thereof an effective amount of a composition comprising a deoxyribonuclease I enzyme (DNase I) conjugated with water soluble polymer, wherein the DNase I conjugate has a systemic clearance at least 50% or lower compared to DNase I that is not conjugated with a water soluble polymer, and wherein the composition is formulated for parenteral administration.

In one embodiment, the disease state is selected from the group consisting of an infection by a pathological microorganism, ischemia, diabetes, atherosclerosis, delayed type hypersensitivity, stroke, cancer, metastatic cancer (pancreatic, lung, hepatoma, and colorectal), acute kidney injury, GVH disease, venous thromboembolism, atherosclerosis, liver failure, acute lung injury and pulmonary fibrosis, dry eye disease, Alzheimer's disease, and disseminated intravascular coagulation.

In one embodiment, the composition is administered subcutaneously, intravenously, intraperitoneally, or intramuscularly. In one embodiment, the composition is not administered by inhalation.

### DESCRIPTION OF THE DRAWINGS

FIG. 1: Concentration of Test Article Equivalents in Whole Blood following a Single IV Dose of DNase.

FIG. 2: Concentration of Test Article Equivalents in Whole Blood following a Single IV, IM, or IP Dose.

FIG. 3: Concentration of Test Article Equivalents in Whole Blood following a Single IV Dose of PSA-DNase.

FIG. 4: Concentration of Test Article Equivalents in Whole Blood following a Single IV, SC, or IP Dose of PSA-DNase 14K.

FIG. 5: Concentration of Test Article Equivalents in Whole Blood following a Single SC Dose—DNase vs. PSA-DNase 14K vs. PSA-DNase 24K.

FIG. 6: Concentration of Test Article Equivalents in Whole Blood following a Single IV Dose—DNase vs. PSA-DNase.

FIG. 7: Concentration of Test Article Equivalents in Whole Blood following a Single IP Dose—DNase vs. PSA-DNase 14K.

FIG. 8: Pharmacokinetics of Test Article Equivalents in Whole Blood Collected from Rats following an Intravenous, Subcutaneous, Intramuscular, or Intraperitoneal Dose

### DETAILED DESCRIPTION OF THE INVENTION

The present invention provides conjugates of deoxyribonuclease enzymes with water soluble polymers such as PSA having improved pharmacokinetic attributes. These modifications provide unexpectedly high levels of DNA hydrolytic activity in blood and other bodily tissues over the time due to markedly increased distribution phase and reduced clearance of DNase conjugates after delivery to blood circulation relative to the unconjugated compounds, while half-life and residence time of conjugates remains almost unchanged compared to the unconjugated DNase compounds. The compositions of the invention are used for parenteral treatment of diseases related to NET formation and the presence of extracellular DNA.

Clearance is a pharmacokinetic measurement of the volume of plasma from which a substance is completely removed per unit time; the usual units are mL/min. The quantity reflects the rate of drug elimination divided by plasma concentration.

The water soluble polymer-DNase conjugates of the invention have systemic clearance in a subject's body at least 50%, 45%, 40%, 35%, 30%, 25%, 20%, 15%, or 10% or lower compared to DNase that is not conjugated with a water soluble polymer.

The volume of distribution (V_{D}, also known as apparent volume of distribution) is the theoretical volume that would be necessary to contain the total amount of an administered drug at the same concentration that it is observed in the blood plasma. It is defined as the distribution of a medication between plasma and the rest of the body after oral or parenteral dosing. The V_{D} of a drug represents the degree to which a drug is distributed in body tissue rather than the plasma. V_{D} is directly correlated with the amount of drug distributed into tissue; a higher V_{D} indicates a greater amount of tissue distribution.

The water soluble polymer-DNase conjugates of the invention have a volume of distribution in a subject's body at least 50%, 45%, 40%, 35%, 30%, 25%, 20%, 15%, or 10% or lower compared to DNase that is not conjugated with a water soluble polymer.

The compositions of the invention are formulated for parenteral administration, e.g., subcutaneous, intravenous, intraperitoneal, and intramuscular administration. Suitable disease states for treatment include pathological microorganism, ischemia, diabetes, atherosclerosis, delayed type hypersensitivity, stroke, cancer, metastatic cancer (pancreatic, lung, hepatoma, and colorectal), acute kidney injury, GVH disease, venous thromboembolism, atherosclerosis, liver failure, acute lung injury and pulmonary fibrosis, dry eye disease, Alzheimer's disease, and disseminated intravascular coagulation. Other disease states are listed in U.S. Pat. Nos. 8,871,200, 8,431,123, 9,072,733, 8,388,951, 8,796,004, 8,535,663, 8,916,151, 8,871,200, 7,612,032, 8,710,012, 9,248,166, 9,463,223, U.S. patent application Ser. Nos. 14/579,041, 15/287,447, 14/988,340, 14/988,340, 15/157,910, and PCT/RU2016/000284, herein each incorporated by reference in their entirety

### DNase Compositions

DNase refers to any enzyme that catalyzes the hydrolytic cleavage of phosphodiester linkages in the DNA backbone, thus degrading DNA. Some DNases cut, or cleave, only residues at the ends of DNA molecules (exodeoxyribonucleasess, a type of exonucleasee). Others cleave anywhere along the chain (endodeoxyribonuclease, a subset of endonucleases). Some DNases are fairly indiscriminate about the DNA sequence at which they cut, while others, including restriction enzymes, are very sequence-specific. In one embodiment, the invention provides DNase I. Hereinafter, when using the term DNase I (or DNase), it also encompasses DNase or DNase I-like proteins. By DNase-like protein is meant a protein which has an activity equivalent to that of DNase. DNase degrades DNA, as detailed above. The activity of DNase or an DNase-like protein can be measured using a standard assay as described in Kunitz (1950). One Kunitz unit is defined as the amount of enzyme added to 1 mg/ml salmon sperm DNA that causes an increase in absorbance of 0.001 per minute at the wavelength of 260 nm when acting upon highly polymerized DNA at 25° C. in a 0.1 M NaOAc (pH 5.0) buffer. Typically, an DNase-like protein has at least 35% of the activity of standard DNase I, and preferably, at least 50% of the activity of standard DNase I.

Mutants of DNase which have the requisite activity, as detailed above, may also be used. An “DNase-like” protein may also be referred to as an “DNase-homologue”. Whether two sequences are homologous is routinely calculated using a percentage similarity or identity, terms that are well known in the art. References sequences for human DNase I include accession numbers AAA63170.1, AAB00495.1, and CAC12813.1.

In this invention, homologues have 50% or greater similarity or identity at the nucleic acid or amino acid level, preferably 60%, 70%, 80% or greater, more preferably 90% or greater, such as 95% or 99% identity or similarity at the amino acid level. A number of programs are available to calculate similarity or identity; preferred programs are the BLASTn, BLASTp and BLASTx programs, run with default parameters (available on the NCBI-NIH database). For example, 2 amino acid sequences may be compared using the BLASTn program with default parameters (score=100, word length=11, expectation value=11, low complexity filtering=on). The above levels of homology may be calculated using these default parameters.

DNase Derivatized with Polysaccharide

As described above, in addition to the naturally occurring or engineered glycosylation pattern of DNase produced by cells expressing DNase, the DNase molecule may be chemically derivatized with a water soluble polymer such as a polysaccharide. Preferably, the polysaccharide has at least 2, more preferably at least 5, most preferably at least 10, for instance at least 50 or more saccharide units.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine). An exemplary polysaccharide is poly(sialic acid). An exemplary poly(ether) is poly(ethylene glycol). Poly(ethylene imine) is an exemplary polyamine, and poly(acrylic) acid is a representative poly(carboxylic acid). The water soluble polymer can be PEG, poly(2-ethyl 2-oxazoline), poly [oligo(ethylene glycol) methyl methacrylate], polyoxazoline, poly(N-(2-hydroxypropyl)) methacrylamide, polyglycerol, poly(N-vinylpyrrolidone), polycarbonate, poly(carboxybetaine methacrylate), poly(sulfobetaine methacrylate) or poly(2-methyacryloyloxyethyl). phosphorylcholine). The polysaccharide is selected from polysialic acid, dextran, dextrin, heparin, hyaluronic acid, hydroxyethyl starch and chondroitin sulphate. Preferably, the polysaccharide is polysialic acid and consists substantially only of sialic acid units. However, the polysaccharide may have units other than sialic acid in the molecule. For instance, sialic acid units may alternate with other saccharide units. Preferably, however, the polysaccharide consists only of units of sialic acid. The polymer backbone can be linear or branched. Branched polymer backbones are generally known in the art. Typically, a branched polymer has a central branch core moiety and a plurality of linear polymer chains linked to the central branch core.

Preferably, the derivatized compound is an N-terminal derivative of DNase or of an DNase-like protein, that is, the polysaccharide is associated with the DNase at its N-terminus. Alternatively, however, the polysaccharide may be associated with the DNase or DNase-like protein at a mid-chain amino acid, such as at the side chain of a lysine, cysteine, aspartic acid, arginine, glutamine, tyrosine, glutamic acid or histidine. Typically, the side chain is of a lysine of cysteine amino acid.

Preferably the polysaccharide has a terminal sialic acid group, and as detailed above, is more preferably a polysialic acid, that is a polysaccharide comprising at least 2 sialic acid units joined to one another through α-2-8 or α-2-9 linkages. A suitable polysialic acid has a weight average molecular weight in the range 2 to 50 kDa, preferably in the range 5 to 50 kDa. Most preferably, the polysialic acid is derived from a bacterial source, for instance polysaccharide B of E. coli KI, Maraxella liquefaciens or Pasteurella aeruginosa or K92 polysaccharide from E. coli K92 strain. It is most preferably colominic acid from E. coli K1.

The polysialic acid may be in the form of a salt or the free acid. It may be in a hydrolysed form, such that the molecular weight has been reduced following recovery from a bacterial source.

The polysaccharide, which is preferably polysialic acid may be material having a wide spread of molecular weights such as having a polydispersity of more than 1.3, for instance as much as 2 or more. Preferably the polydispersity (p.d.) of molecular weight is less than 1.3, more preferably less than 1.2, for instance less than 1.1. The p.d. may be as low as 1.01.

The DNase may be derivatised with more than one anionic polysaccharide. For instance, the DNase may be derivatised at both its N-terminus and at an internal amino acid side chain. The side chains of lysine, cysteine, aspartic acid, arginine, glutamine, tyrosine, glutamic acid, serine and histidine, for instance, may be derivatised by an anionic polysaccharide. The DNase may also be derivatised on a glycon unit. However, in a preferred embodiment of this invention, the DNase is derivatised at its N-terminus only.

The derivatized compound may be a covalently-linked conjugate between the DNase and an anionic polysaccharide. The DNase may be covalently linked to the polysaccharide at its N-terminal amino acid. The covalent linkage may be an amide linkage between a carboxyl group and an amine group. Another linkage by which the DNase could be covalently bonded to the polysaccharide is via a Schiff base. Suitable groups for conjugating to amines are described further in WO2006/016168. The DNase can be conjugated to the polysaccharide via a reactive aldehyde on the polysaccharide. Chemistry suitable for preparing a polysaccharide with a reactive aldehyde at the reducing terminal of a polysaccharide is described in WO 05/016974. The process involves a preliminary selective oxidation step followed by reduction and then further oxidation to produce a compound with an aldehyde at the reducing terminal and a passivated non-reducing end.

Suitable linkers are derived from N-maleimide, vinylsulphone, N-iodoacetamide, orthopyridyl or N-hydroxysuccinimide-containing reagents. The linker may also be biostable or biodegradable and comprise, for instance, a polypeptide or a synthetic oligomer. The linker may be derived from a bifunctional moiety, as further described in WO2005/016973. A suitable bifunctional reagent is, for instance, Bis-NHS.

### Pharmaceutical Compositions and Administration

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the DNase molecule disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

The pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

Further, the composition may be formulated into a single dosage form suitable for the patient's body, and preferably is formulated into a preparation useful for protein drugs according to the typical method in the pharmaceutical field so as to be administered by an oral or parenteral route such as through skin, intravenous, intramuscular, intra-arterial, intramedullary, intramedullary, intraventricular, transdermal, subcutaneous, intraperitoneal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The pharmaceutical composition disclosed herein is expected to have longer in vivo duration of efficacy and titer, thereby remarkably reducing the number and frequency of administration thereof.

Moreover, the pharmaceutical composition may be administered alone or in combination or coincident with other pharmaceutical formulations showing prophylactic or therapeutic efficacy.

The therapeutic method of the present specification may include the step of administering the composition including the DNase protein at a pharmaceutically effective amount. The total daily dose should be determined through appropriate medical judgment by a physician, and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg, at least 0.25 mg, at least 0.3 mg, at least 0.5 mg, at least 0.75 mg, at least 1 mg, at least 1.25 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 50 mg, at least 55 mg, at least 60 mg, at least 65 mg, or at least 70 mg. In one embodiment, a weekly dose may be at most 0.5 mg, at most 0.75 mg, at most 1 mg, at most 1.25 mg, at most 1.5 mg, at most 2 mg, at most 2.5 mg, at most 3 mg, at most 4 mg, at most 5 mg, at most 6 mg, at most 7 mg, at most 8 mg, at most 9 mg, at most 10 mg, at most 15 mg, at most 20 mg, at most 25 mg, at most 30 mg, at most 35 mg, at most 40 mg, at most 50 mg, at most 55 mg, at most 60 mg, at most 65 mg, or at most 70 mg. In a particular aspect, the weekly dose may range from 0.25 mg to 2.0 mg, from 0.5 mg to 1.75 mg. In an alternative aspect, the weekly dose may range from 10 mg to 70 mg.

### Definitions

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The present composition encompasses amino acid substitutions in proteins and peptides, which do not generally alter the activity of the proteins or peptides (H. Neurath, R. L. Hill, The Proteins, Academic Press, New York, 1979). In one embodiment, these substitutions are “conservative” amino acid substitutions. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Analogue as used herein denotes a peptide, polypeptide, or protein sequence which differs from a reference peptide, polypeptide, or protein sequence. Such differences may be the addition, deletion, or substitution of amino acids, phosphorylation, sulfation, acrylation, glycosylation, methylation, farnesylation, acetylation, amidation, and the like, the use of non-natural amino acid structures, or other such modifications as known in the art.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the term “prevention” means all of the actions by which the occurrence of the disease is restrained or retarded.

As used herein, the term “treatment” means all of the actions by which the symptoms of the disease have been alleviated, improved or ameliorated. In the present specification, “treatment” means that the symptoms of multiple myeloma are alleviated, improved or ameliorated by administration of the DNase proteins disclosed herein.

As used herein, the term “administration” means introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, or rectal administration. However, since peptides are digested upon oral administration, active ingredients of a composition for oral administration should be coated or formulated for protection against degradation in the stomach.

In the present specification, the term “subject” is those suspected of having multiple myeloma. However, any subject to be treated with the DNase proteins or the pharmaceutical composition disclosed herein is included without limitation. The subject is being treated with DNase to inhibit the primary cancer and not as a treatment for anemia related to the disease state or to administration of chemotherapy.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1

### Pk Comparison of Single Dosing of Labeled DNase and PSA-DNase

Summary of Phase I and Phase II

The objective of phase 1 of this study was to determine and compare the pharmacokinetics of test article-derived radioactivity after a single intravenous (IV, bolus) or subcutaneous (SC) dose of 125I-DNase (100L-SUB-3385-098-003) or 125I-PSA-DNase [14K (9-65) RA] to male Sprague Dawley rats. Urine was also collected over 24 hours post-dosing from an additional 3 rats receiving IV or SC 125I-DNase (100L-SUB-3385-098-003) or 125I-PSA-DNase [14K (9-65) RA].

The objective of phase 2 of this study was to determine and compare the pharmacokinetics of test article-derived radioactivity after a single dose of 125I-DNase (100L-SUB-3385-098-003, 250L-SUB-3385-098-001 or 13001) or 125I-PSA-DNase [14K (9-65) RA, 14K 050614 or 24K (9-66) RA] administered by IV, intraperitoneal (IP), or intramuscular (IM) routes. Tissues were collected at 24 hours post-dosing from 3 rats receiving IV, IM, or IP 125I-DNase, IV or IP 125I-PSA-DNase 14K, or IV or SC 125I-PSA-DNase 24K.

Phase 1: Four dose groups of 6 male rats each were designated for blood collection. Two groups received a single IV (bolus) dose of 125I-DNase or 125I-PSA-DNase 14K (9-65) RA. Following IV (bolus) dosing, blood samples were collected from 3 animals per group at 9 time-points over 32 hours post-dosing. Two groups received a single SC dose of 125I-DNase or 125I-PSA-DNase. Following SC dosing, blood samples were collected from 3 animals per group at 10 time-points over 32 hours post-dosing.

Four dose groups of 3 male rats each were designated for urine collection. Two groups received a single IV (bolus) dose and 2 groups received a single SC dose of 125I-DNase or 125I-PSA-DNase. Following dosing, animals were placed into individual metabolism cages for collection of urine over 24 hours. Blood and urine were precipitated with TCA to determine the fraction of 125I that was bound to the test article.

Phase 2: Nine dose groups of 6 male rats each received a single dose of 125I-DNase or 125I-PSA-DNase. Five groups received an IV, IP, or IM dose of 125I-DNase, 2 groups received an IV or IP dose of 125I-PSA-DNase 14K, and 2 groups received an IV or SC dose of 125I-PSA-DNase 24K. Following dosing, blood samples were collected from 3 animals per group at 9 time-points. At the completion of the blood collection for the animals scheduled for the 24 hour post-dose time-point, animals were euthanized by inhalation of CO2 and the liver, kidney, spleen, lung, trachea, and bronchus were collected.

Summary of Results: Concentration, specific activity, and all downstream calculations were performed on PSA-DNase as DNase equivalents (equal masses of protein moiety and consequently equal molar concentrations of DNase regardless of conjugation) to simplify comparison across dose groups; the target dose was 0.1 mg/kg protein. The actual dose administered to each group, as DNase or DNase equivalents, ranged from 0.14 to 0.53 mg/kg.

Following an IV (bolus) dose of 125I-DNase, the whole blood concentration vs time profile was comparable among the lots tested. The mean of maximal concentrations (mean of Cmax) of test article equivalents in whole blood was 1.540 μg/g at approximately 5 minutes post-dosing (the first collection time point and Tmax). 125I-DNase exhibited biphasic behavior, i.e., an initial rapid decline in concentration (distribution phase) over the first 2 hours post-dosing followed by a slower terminal elimination phase. The mean dose-normalized AUClast was 7.935 (h·μg/g)/(mg/kg). The mean terminal elimination half-life was 5.1 hours.

Administration of 125I-DNase by SC, IM, and IP routes resulted in similar whole blood time courses for DNase equivalents. Overall whole blood exposure (mean dose-normalized AUClast) was 2.76 (h·μg/g)/(mg/kg), 4.25 (h·μg/g)/(mg/kg), and 7.20 (h·μg/g)/(mg/kg), respectively, which is less than exposure measured following IV administration. Tmax was 1.5 to 2 hours. DNase bioavailability (F) was approximately 43.3% for SC dosing, 42.6% for IM dosing, and 72.1% for IP dosing.

After IV administration, 125I-PSA-DNase showed similar biphasic behavior to unconjugated DNase, i.e., an initial rapid decline in concentration (distribution phase) over the first one hour post-dosing followed by a slower terminal elimination phase. However, up to a 3-fold difference in the dose-normalized AUClast was apparent between different PSA-DNase conjugates in a phase and lot-dependent manner. The mean Cmax for 125I-PSA-DNase 14K was 1.858 μg/g, measured at the first time point of 5 minutes post dosing (Tmax). Dose-normalized AUClast was 20 (h·μg/g)/(mg/kg); although lot/phase difference was evident, the overall time courses were very similar. The mean terminal elimination half-life was 3.1 hours. In comparison, Cmax for 125I-PSA-DNase 24K at 5 minutes post dosing (Tmax) was 2.252 μg/g, AUClast was 40.7 (h·μg/g)/(mg/kg) and terminal elimination half-life was 5.45 hours.

Whole blood exposure to 125I-PSA-DNase 14K was substantially less following SC administration versus IV administration. Subcutaneous bioavailability (F) was 18.1% for 125I-PSA-DNase 14K, Tmax was 6 hours, and dose-normalized AUClast was 2.54 (h·μg/g)/(mg/kg). After IP administration, Tmax was 2 hours, dose-normalized AUClast was 17.9 (h·μg/g/(mg/kg), and bioavailability was 128%. 125I-PSA-DNase 14K is highly bioavailable when administered IP; exposure greater than that after IV administration is likely an artifact of the sparse sampling design and dose route.

Whole blood exposure to 125I-PSA-DNase 24K was substantially less following SC administration versus IV administration. Subcutaneous bioavailability (F) was 13.9% for 125I-PSA-DNase 24K, Tmax was 8 hours, and dose-normalized AUClast was 5.64 (h·μg/g)/(mg/kg). IP administration of 125I-PSA-DNase 24K was not attempted.

Following IV dosing, the mean terminal elimination half-life for 125I-DNase and 125I-PSA-DNase appeared to be similar but overall exposure was lower for DNase; this difference appeared to be dictated by the extent of distribution, as indicated by the 3.5- and 5-fold greater mean Vss for 125I-DNase versus 125I-PSA-DNase 14K and 125I-PSA-DNase 24K, respectively. Following SC dosing, DNase and PSA-DNase had similar whole blood concentration profiles and overall exposure, although DNase articles appeared to have a longer and more sustained absorption phase than PSA-DNase.

Following IV dosing, ratio between mean exposure for 125I-DNase, 125I-PSA-DNase 14K, and 125I-PSA-DNase 24K test articles was 1:2.5:5.1, respectively. Following IP dosing, exposure was similar to IV dosing and 125I-PSA-DNase 14K exposure was higher than exposure to 125I-DNase. DNase exposure following an IM dose was greater than SC dosing and less than IV or IP dosing; no IM dosing of any PSA-DNase was attempted.

Trichloroacetic acid (TCA) precipitation of whole blood samples collected after IV dosing of 125I-DNase and 125I-PSA-DNase indicated that approximately 94% of the radioactivity in the whole blood collected from 5 minutes post-dosing to 2 hours post-dosing was associated with the pellet fraction following centrifugation and, therefore, was still bound to the test article. Approximately 85% of the radioactivity in the whole blood collected following SC dosing was associated with the pellet fraction following centrifugation.

Test article equivalents were detected in kidney, liver, spleen, and lung 24 hours after administration of 125I-DNase or 125I-PSA-DNase to Groups 10 through 18. The concentration of test article equivalents in the bronchus was generally below the LLOQ for all groups (1 animal in Group 17 was marginally above the LLOQ). The concentration in the liver relative to the kidney was highest after IV administration of 125I-PSA-DNase 24K, followed by 125I-PSA-DNase 14K; tissue concentrations normalized to administered dose and blood concentration show the same trend. No definitive trends in tissue concentration were apparent relative to the test article administered (DNase or conjugated DNase), or the dose route.

Following IV or SC dosing, urine was collected from 3 animals each in Group 5, Group 6, Group 7, and Group 8 over 24 hours post-dosing. The majority of the administered dose (approximately 75% to 86%) was accounted for in urine. The dose route and the conjugation of the DNase had no impact on the extent of urinary excretion. The majority of the radioactivity in the urine was located in the supernatant fraction after TCA precipitation, which suggests that it was not bound to the test article.

Throughout the study, all dose groups received a target dosage of 0.1 mg/kg DNase or DNase equivalents. DNase equivalents are the concentration or dosage of conjugated DNase calculated based on the mass of protein moiety alone. Thus, PSA-DNase, 125I-PSA-DNase, and 125I-DNase formulations were prepared to contain an equivalent amount of DNase regardless of conjugation. Serum and tissue concentrations are also presented in DNase equivalents and, thus, can be directly compared across dose groups.

Concentration and Kinetics in Whole Blood

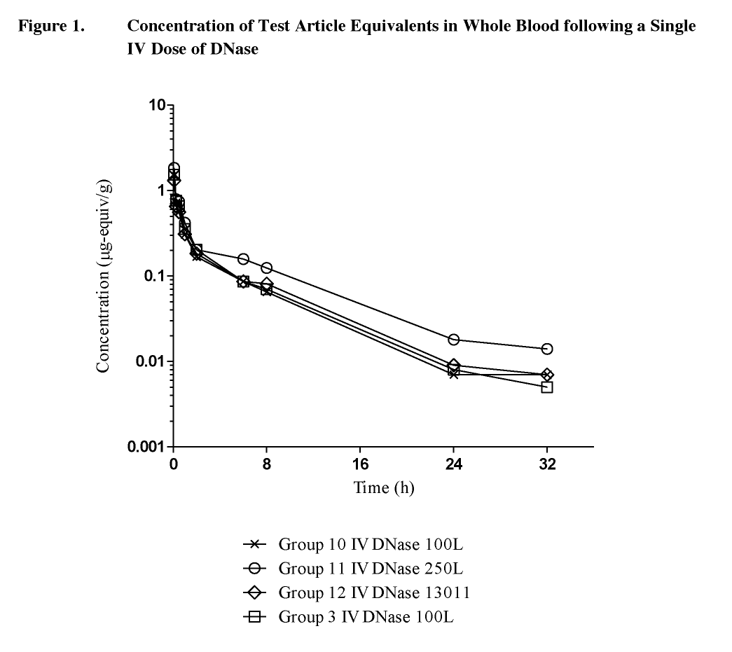

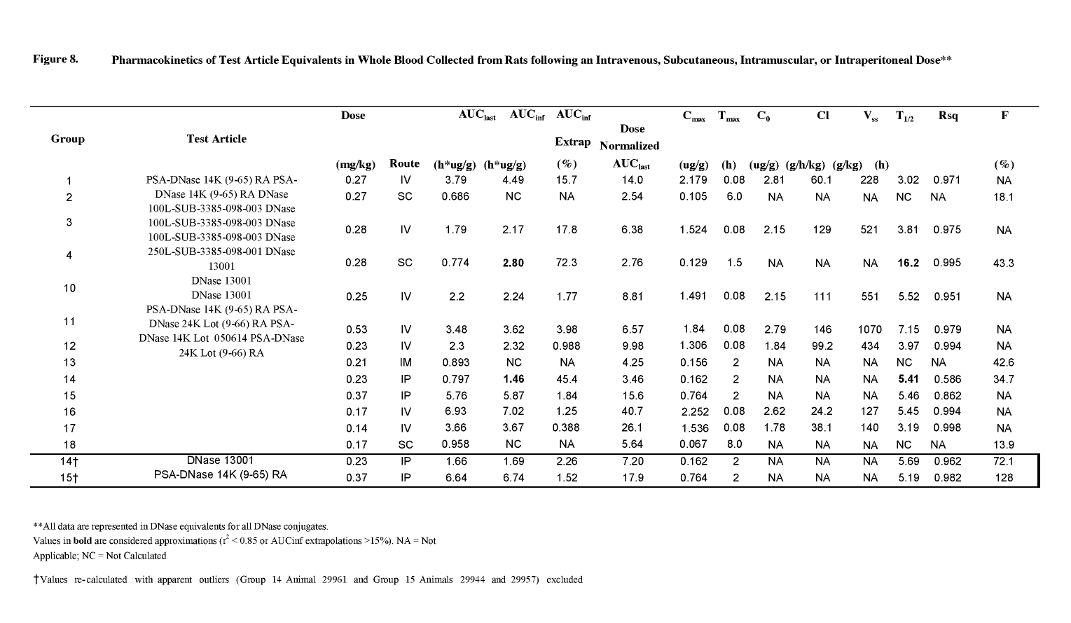

DNase: The mean concentrations of [125I]-test article equivalents in whole blood collected from rats in each dose group are illustrated in FIG. 1 through FIG. 7. Pharmacokinetics of [125I]-test article equivalents in whole blood are shown in FIG. 8. Following an IV (bolus) dose of 125I-DNase, the whole blood concentration vs time profile was comparable among the lots tested. The mean of maximal concentration (mean of Cmax) of test article equivalents in whole blood was highest at the first sampling time-point, approximately 5 minutes post-dosing (Group 3, Group 10, Group 11, Group 12 [FIG. 1]). The mean of Cmax at this time point was approximately 1.540 μg/g; the mean extrapolated concentration at the moment of injection (C0) was 2.233 μg/g. 125I-DNase exhibited a biphasic behavior, i.e., an initial rapid decline in concentration (distribution phase) over the first 2 hours post-dosing followed by a slower terminal elimination phase. The dose-normalized AUClast was relatively constant among the 3 lots of 125I-DNase, with values ranging from 6.57 (h·μg/g)/(mg/kg) to 9.98 (h·μg/g)/(mg/kg) in Phase II (Group 10, Group 11, and Group 12), which agrees with the 125I-DNase dose-normalized AUClast of 6.38 (h·μg/g)/(mg/kg) in Phase I (Group 3). The mean dose-normalized AUClast was 7.935 (h·μg/g)/(mg/kg). The mean terminal elimination half-life was 5.1 hours.

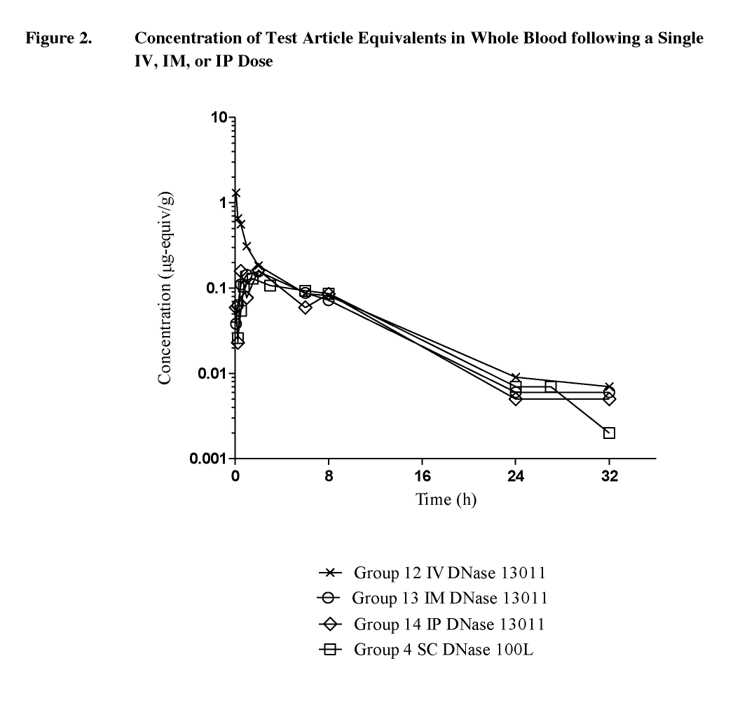

Following SC, IM, and IP administration of 125I-DNase (Group 4, Group 13, and Group 14, respectively), similar whole blood time courses were seen for DNase equivalents (FIG. 2). After IP administration (Group 14), mean concentrations were inconsistent over the time course and further examination indicated that 1 animal in Group 14 had low whole blood concentrations that may have been associated with dose administration in abdominal fat or tissues; this animal was excluded from the IP dosing pharmacokinetic calculations. Tmax was 1.5 to 2 hours, mean dose-normalized AUClast was 2.76 (h·μg/g)/(mg/kg), 4.25 (h·μg/g)/(mg/kg), and 7.20 (h·μg/g)/(mg/kg), and bioavailability (F) was 43.3%, 42.6% and 72.1% for SC, IM, and IP dosing, respectively. No distinct difference was apparent among these routes of administration.

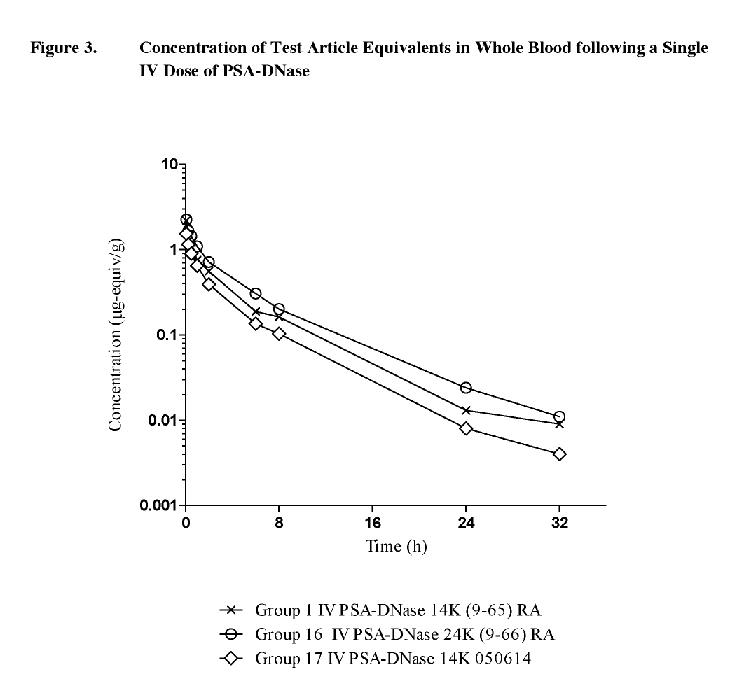

DNase-PSA: Following an IV (bolus) dose of 125I-PSA-DNase, Tmax was the first sampling time-point, approximately 5 minutes post-dosing, regardless of the molecular weight of the conjugate (Group 1, Group 16, and Group 17 [FIG. 3]). 125I-PSA-DNase exhibited a similar biphasic behavior as 125I-DNase; the overall time courses for different 125I-PSA-DNase treatments were very similar.

Following IV administration of 125I-PSA-DNase 14K (Group 1 and Group 17), the mean Cmax was 1.858 μg/g, and the mean extrapolated C0 was 2.295 μg/g. The mean dose-normalized AUClast was 20 (h·μg/g)/(mg/kg), although differences were evident between 125I-PSA-DNase 14K (9-65) RA in Phase I (14.0 (h·μg/g)/(mg/kg), Group 1) and 125I-PSA-DNase 14K 050614 (Group 17) in Phase II (26.1 (h·μg/g)/(mg/kg)). The mean terminal elimination half-life was 3.1 hours.

Following IV administration of 125I-PSA-DNase 24K (Group 16), Cmax was 2.252 μg/g, AUClast was 40.7 (h·μg/g)/(mg/kg), and terminal elimination half-life was 5.45 hours.

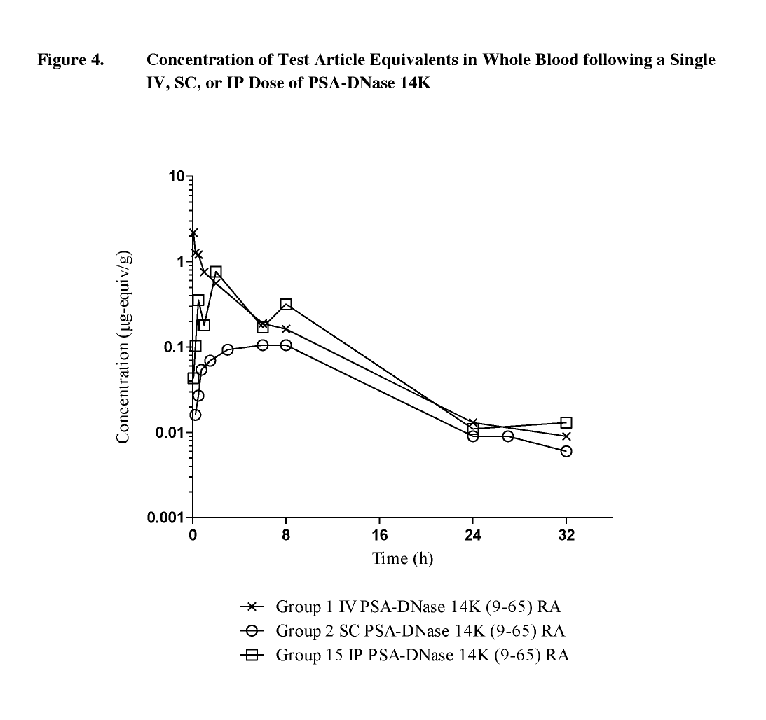

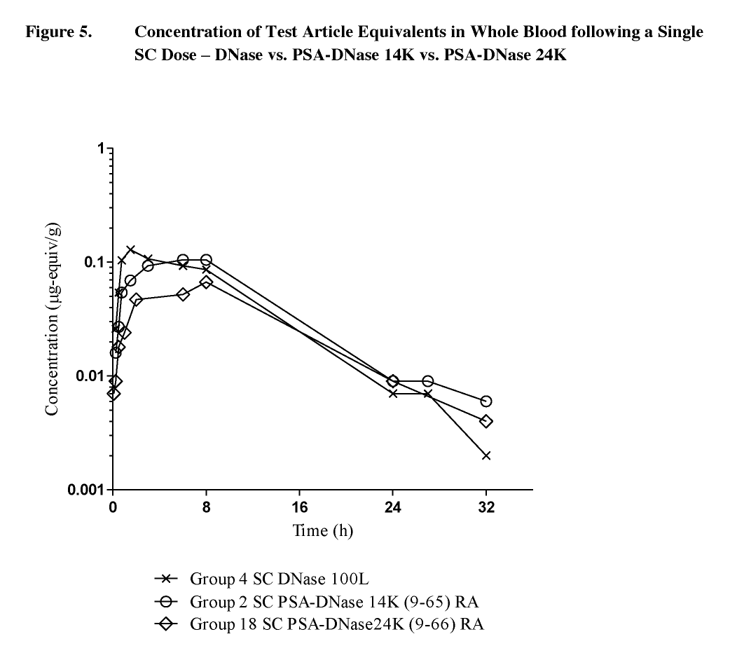

Following SC administration of 125I-PSA-DNase 14K (Group 2 [FIG. 4]), Tmax was 6 hours, dose-normalized AUClast was 2.54 (h·μg/g)/(mg/kg), and bioavailability was 18.1%. After IP administration (Group 15 [FIG. 4]), mean concentrations were inconsistent over the time course and further examination indicated that 2 animals in Group 15 had low whole blood concentrations that may have been associated with dose administration in abdominal fat or tissues; these animals were excluded from the IP dosing pharmacokinetic calculations. For the remaining animals, Tmax was 2 hours, dose-normalized AUClast was 17.9 (h·μg/g)/(mg/kg); and bioavailability was 128%. Bioavailability indicates complete systemic availability; the excursion from 100% bioavailability is likely an artifact of the sparse sampling design and dose route. IM administration of 125I-PSA-DNase 14K was not attempted. Following SC administration of 125I-PSA-DNase 24K (Group 18 [FIG. 5]), Tmax was 8 hours, dose-normalized AUClast was 5.64 (h·μg/g)/(mg/kg), and bioavailability was 13.9%. Neither IP nor IM administration of 125I-PSA-DNase 24K was attempted.

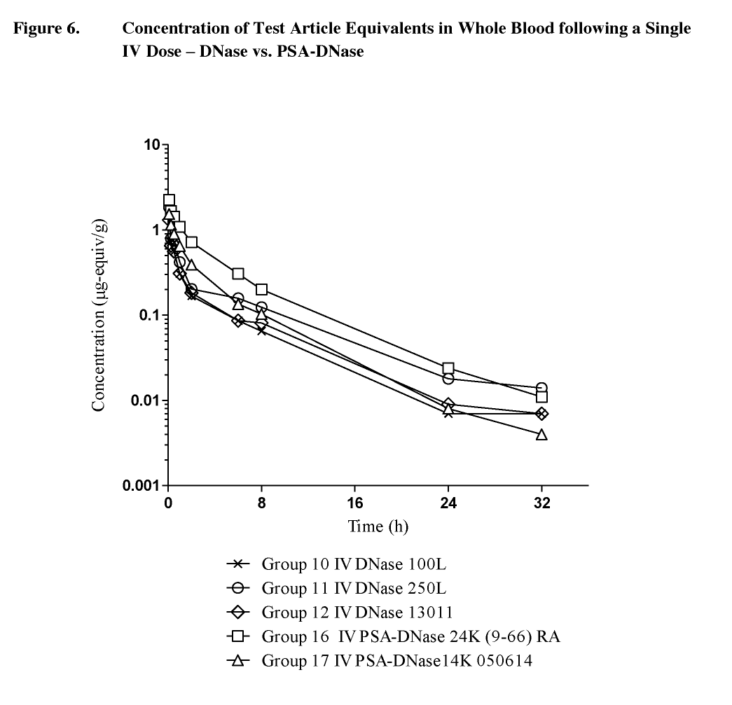

Comparision of DNase and PSA-DNase: Although the pharmacokinetics for 125I-DNase and 125I-PSA-DNase were similar, several differences were evident. Upon IV administration, the mean dose-normalized AUClast was 7.935 (h·μg/g)/(mg/kg) for 125I-DNase, 20 (h·μg/g)/(mg/kg) for 125I-PSA-DNase 14K, and 40.7 (h·μg/g)/(mg/kg) for 125I-PSA-DNase 24K test articles (approximate ratio of 1:2.5:5.1). Because the mean terminal elimination half-lives were similar, the difference in exposure was attributed largely to the extent of the distribution phase (FIG. 6). The duration of the distribution phase appears to be the same for both DNase and PSA-DNase—approximately 1 to 2 hours. However, the extent of distribution is greater for DNase, which results in a lower whole blood concentration at each time point and greater volume of distribution; mean Vss across all tested groups and test articles was approximately 644 g/kg for 125I-DNase, 184 g/kg for 125I-PSA-DNase 14K and 127 g/kg for 125I-PSA-DNase 24K (approximate ratio 5:1.5:1). The mean calculated clearances also reflect the difference, with approximate mean values of 121 g/h/kg for 125I-DNase, 49 g/h/kg for 125I-PSA-DNase 14K, and 24 g/h/kg for 125I-PSA-DNase 24K (approximate ratio 5:2:1). The general fold differences in several pharmacokinetic parameters between DNase, PSA-DNase 14K, and PSA-DNase 24K indicate a slight trend toward a longer distribution phase and slower clearance of the conjugated material with tendency being potentiated by the size of conjugated PSA, but the difference is small and not remarkably different than that expected from normal variability.

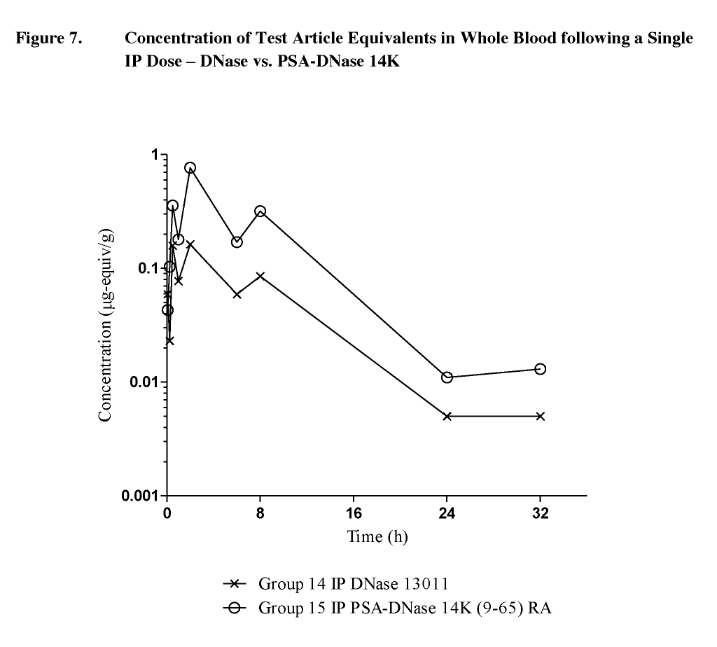

Following SC administration, 125I-PSA-DNase 14K appears to have a longer and more sustained absorption phase than 125I-DNase (FIG. 5). As a consequence of differences in IV distribution and SC absorption, SC bioavailability was substantially lower for 125I-PSA-DNase test articles than for DNase. Intraperitoneal dosing of 125I-PSA-DNase 14K resulted in a 4.5-fold higher exposure than IP dosing of 125I-DNase (FIG. 7).

Summary: Following an IV (bolus) dose of 125I-DNase, 125I-DNase exhibited biphasic behavior, i.e., an initial rapid decline in concentration (distribution phase) over the first 2 hours post-dosing followed by a slower terminal elimination phase. The dose-normalized AUClast was relatively constant among the 3 lots of DNase tested; the mean dose-normalized AUClast was 7.935 (h·μg/g)/(mg/kg). The mean terminal elimination half-life was 5.1 hours. Following SC, IM, and IP administration of 125I-DNase, similar whole blood time courses were seen for DNase equivalents. Tmax was 1.5 to 2 hours, mean dose-normalized AUClast was 2.76 (h·μg/g)/(mg/kg), 4.25 (h·μg/g)/(mg/kg), and 7.20 (h·μg/g)/(mg/kg), and bioavailability (F) was 43.3%, 42.6% and 72.1% for SC, IM, and IP dosing, respectively.

Following an IV (bolus) administration, 125I-PSA-DNase exhibited biphasic behavior similar to that of 125I-DNase; the overall time courses were very similar.

The mean dose-normalized AUClast for 125I-PSA-DNase 14K was 20 (h·μg/g)/(mg/kg); although phase-to-phase differences were evident. The mean terminal elimination half-life was 3.0 hours. Following SC administration of 125I-PSA-DNase 14K, Tmax was 6 hours, dose-normalized AUClast was 2.54 (h·μg/g)/(mg/kg), and bioavailability was 18.1%. After IP administration, Tmax was 2 hours, dose-normalized AUClast was 17.9 (h·μg/g)/(mg/kg), and bioavailability was 128%.

Following IV administration, the dose-normalized AUClast for 125I-PSA-DNase 24K was 40.7 (h·μg/g)/(mg/kg), the terminal elimination half-life was 5.45 hours. Following SC administration of 125I-PSA-DNase 24K, Tmax was 8 hours, dose-normalized AUClast was 5.64 (h·μg/g)/(mg/kg), and bioavailability was 13.9%. IP administration was not attempted. No notable difference in whole blood Cmax, C0, or the terminal elimination half-life was evident between 125I-DNase and 125I-PSA-DNase 14K when administered intravenously to rats in Phase I, but a 2-fold difference in overall exposure as measured by the dose-normalized AUClast was apparent (6.38 (h·μg/g)/(mg/kg) for DNase versus 14.0 (h·μg/g)/(mg/kg) for 125I-PSA-DNase 14K). The same trend was evident in Phase II, but the difference was amplified by an apparent difference in dose-normalized AUClast between lots of 125I-PSA-DNase conjugated with PSA of different molecular weight, with no similar change in exposure for different lots of DNase. The dose-normalized AUClast for intravenously administered PSA-DNase 14K originating from different lots ranged from approximately 14.0 (h·μg/g)/(mg/kg) to 26.1 (h·μg/g)/(mg/kg). The difference in exposure between the DNase and PSA-DNase test articles was attributed to the longer distribution phase for DNase, resulting in a lower whole blood concentration at each time point and greater volume of distribution; mean Vss was 644 g/kg for DNase, 184 g/kg for 125I-PSA-DNase 14K and 127 g/kg for PSA-DNase 24K.

The difference in mean exposures following an IP dose of 125I-DNase and 125I-PSA-DNase 14K were approximately 1:4.5. The difference in mean exposures following an IV dose of 125I-DNase, 125I-PSA-DNase 12K and 125I-PSA-DNase 24K were approximately 1:2.5:5.1. Conversely, following an SC dose no significant difference in exposure was observed between 125I-DNase and 125I-PSA-DNase 14K in phase I and between DNase and PSA-DNase 24K in phase II, although 125I-PSA-DNase appears to have a longer and more sustained absorption phase than 125I-DNase. 125I-DNase exposure following an IM dose was similar to that following an IP dose; no IM dosing of PSA-DNase was attempted.

The majority of the radiolabel in whole blood appears to be bound to the test article. After IV administration, the percentage of radioactivity in the whole blood associated with the pellet fraction after TCA precipitation was approximately 94% for both 125I-DNase and 125I-PSA-DNase. After SC administration, it was approximately 83% and 86% for 125I-DNase and 125I-PSA-DNase, respectively.

Test article equivalents were detected in kidney, liver, spleen, and lung 24 hours after administration of 125I-DNase or 125I-PSA-DNase. The highest concentrations in tissue were noted after IV administration of PSA-DNase 24K followed by PSA-DNase 14K, consistent with the whole blood exposure for these lots versus other dose groups. Tissue concentrations were high after IV administration of DNase in Group 11, but this group received the highest dose of all groups (0.53 mg/kg) and did not have a correspondingly high dose-normalized AUClast. The concentration in the liver relative to the kidney was highest after IV administration of PSA-DNase 24K versus DNase or any other route of exposure. No other trend in tissue concentration was apparent relative to the test article administered (DNase or PSA-DNase), or the dose route.

Following IV or SC dosing of PSA-DNase or DNase, approximately 75% to 86% of the administered radioactivity was recovered in urine over 24 hours post-dosing. The majority of this radioactivity (approximately 68% to 83%) was found in the supernatant after TCA precipitation, which suggests that it was not bound to the test article. The dose route and the conjugation of the DNase had no apparent impact on the extent of urinary excretion.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A therapeutic composition for enzymatic cleavage of circulating cell free DNA and neutrophil extracellular traps in blood, the composition comprising a deoxyribonuclease enzyme conjugated with a water soluble polymer, wherein the DNase I conjugate has a systemic clearance and apparent volume of distribution each at least 50% or lower as compared to DNase that is not conjugated with a water soluble polymer; and wherein the DNase is formulated for parenteral administration.

2. The composition of claim 1, wherein the DNase is DNase I.

3. The composition of claim 1, wherein the DNase is conjugated to the water soluble polymer via a linking group.

4. The composition of claim 1, wherein water soluble polymer is PEG, poly(2-ethyl 2-oxazoline), poly[oligo(ethylene glycol) methyl methacrylate], polyoxazoline, poly(N-(2-hydroxypropyl) methacrylamide, polyglycerol, poly(N-vinylpyrrolidone), polycarbonate, poly(carboxybetaine methacrylate), poly(sulfobetaine methacrylate) or poly(2-methyacryloyloxyethyl phosphorylcholine).

5. The composition of claim 1, wherein the DNase is linked via an amine group at the N-terminus to a water soluble polymer comprising a polysaccharide.

6. The composition of claim 5, wherein the polysaccharide is selected from polysialic acid, heparin, dextran, dextrin, hydroxyethyl starch, hyaluronic acid or chondroitin sulphate.

7. The composition of claim 6, wherein the polysaccharide is polysialic acid.

8. The composition of claim 7, wherein the polysialic acid is attached to the N-terminus of DNase at the reducing terminal unit of the polysialic acid.

9. The composition of claim 2, wherein the DNase I has at least 95% sequence identity to an amino acid sequence comprising Accession No. AAA63170.1, AAB00495.1 or CAC12813.1.

10. The composition of claim 7, wherein the DNase I has an amino acid sequence comprising Accession No. AAA63170.1, AAB00495.1 or CAC12813.1.

11. The composition of claim 2, wherein the DNase I has an amino acid sequence change in DNA binding domain leading to increased hydrolytic activity.

12. The composition of claim 2,wherein the DNase I has an amino acid sequence change in actin binding site leading to loss of actin inhibitory properties

13. A method for treating a disease state associated with circulating cell free DNA and neutrophil extracellular traps in blood, lymph and synovial fluids, the method comprising parenteral administration to a subject in need thereof an effective amount of a composition comprising a deoxyribonuclease I enzyme (DNase I) conjugated with water soluble polymer, wherein the DNase I conjugate has a systemic clearance at least 50% or lower compared to DNase I that is not conjugated with a water soluble polymer, and wherein the composition is formulated for parenteral administration.

14. The method of claim 13, wherein the disease state is selected from the group consisting of an infection by a pathological microorganism, ischemia, diabetes, atherosclerosis, delayed type hypersensitivity, stroke, cancer, metastatic cancer (pancreatic, lung, hepatoma, and colorectal), acute kidney injury, GVH disease, venous thromboembolism, atherosclerosis, liver failure, acute lung injury and pulmonary fibrosis, dry eye disease, Alzheimer's disease, and disseminated intravascular coagulation.

15. The method of claim 13, wherein the composition is administered subcutaneously, intravenously, intraperitoneally, or intramuscularly.

16. The method of claim 13, wherein the composition is not administered by inhalation.
